(19) United States
(12) Patent Application Publication
(10) Pub. No.: US 2016/0311691 A1
(43) Pub. Date: Oct. 27, 2016

(54) METHOD FOR THE PRODUCTION OF LINEAR, CYCLIC AND/OR CAGE-TYPE PERHALOGENATED OLIGOSILYL AND POLYSILYL ANIONS

(71) Applicant: Johann Wolfgang Goethe-Universitat, Frankfurt am Main (DE)

(72) Inventors: Matthias WAGNER, Niddatal (DE); Jan TILLMANN, Frankfurt am Main (DE); Norbert AUNER, Glashütten (DE); Lioba MEYER, Frankfurt am Main (DE); Max HOLTHAUSEN, Oberursel (DE); Hans-Wolfram LERNER, Oberursel (DE)

(21) Appl. No.: 15/105,695

(22) PCT Filed: Dec. 12, 2014
(86) PCT No.: PCT/DE2014/100446

§ 371 Date: Jun. 17, 2016

(30) Foreign Application Priority Data
Dec. 19, 2013 (DE) 10 2013 021 306.1

(51) Int. Cl. C01B 33/107; C07C 211/03; C07C 211/09; H01B 1/12; C08G 77/60

(52) U.S. Cl. CPC C01B 33/107; H01B 1/124; C08G 77/60; C07C 211/09; C07C 211/03

(57) ABSTRACT

The present invention relates to a process for the preparation of linear, cyclic and/or cage-type perhalogenated oligosilyl and polysilyl anions by reacting perhalogenated monosilanes, oligosilanes or polysilanes with organosubstituted ammonium and/or phosphonium halides at temperatures ranging from −80° C. to 85° C., preferably −80° C. to 60° C., and to oligosilyl and polysilyl anions prepared according to that process.

### TECHNICAL FIELD

The present invention relates to a process for the preparation of linear, cyclic and/or cage-type perhalogenated oligosilyl and polysilyl anions and to oligosilyl and polysilyl anions prepared according to that process.

### BACKGROUND

Perhalogenated oligosilane and polysilane compounds form the basis of a wide variety of material-orientated applications, such as the production of amorphous silicon, conductive polymers, silicon layers or also hydrogen storage media, which for their part are of great importance in many fields of industry.

Several processes for preparing oligosilane and polysilane compounds are known in the state of the art. Perhalogenated polysilanes can, for example, be prepared from tetrahalogen silanes and silicon by means of thermal reactions at high temperatures of several 100° C. It is, however, often the case that in this way mixtures of perhalogenated polysilanes with high proportions of short-chain, branched and cyclic compounds are obtained. Furthermore, the production process means that the mixtures obtained are often contaminated with other compounds, which are required for the synthesis.

In DE 10 2005 024 041 A1, for example, a halogen silane and silicon are reacted, generating a plasma discharge, to form a halogenated polysilane, which is subsequently decomposed into silicon in a second step with heating. Spectroscopic examinations have shown that in addition to linear halogenated polysilanes, branched halogenated polysilanes are also obtained after the first synthesis step.

DE 10 2009 056 437 A1, on the other hand, relates to a process for the preparation of short-chain halogenated oligosilanes of the formula Si₂Xₙ₊₂ (n=2-6) and silicon subchlorides SiClₓ (x<2) by the thermolytic decomposition of long-chain halogenated polysilanes. The disadvantages of this process, however, are the high energy input required in order to generate a thermal plasma and the complex processing of the product mixtures obtained in order to isolate the pure products.

A less complex process for preparing the cyclic polysilyl anion [Si₆Cl₁₄]²⁻ is described in WO 2011/094191 A1. Proceeding from trichlorosilane, the anion is obtained by reaction with a tertiary polyamine ligand, or more precisely an alkyl-substituted polyalkylene polyamine, and a deprotonating agent, such as a tertiary amine.

Derivatisation using the Si—Cl functionality is described in WO 2009/148878 A2. In addition, a halogenated polysilane as a pure compound or mixtures of compounds and a plasma chemical process for preparing them are described. This process, however, leads exclusively to hexasilane or its Si-substituted derivatives with comparatively low yields.

W. Lerner et al., Inorganic Chemistry, 51, 8599-8606 (2012), describe a reaction in which dissolving an Si₂Cl₆.TMEDA adduct in dichloromethane leads to the formation of the perchlorinated oligosilanes and polysilanes SiₙCl₂ₙ (n=4, 6, 8, 10) and the chloro-complexed dianion [SiₙCl₂ₙ₊₂]²⁻(n=6, 8, 10, 12).

DE 31 26 240 C2 relates to a process for the preparation of metallic silicon, in which the reaction of perchlorosilane, such as Si₂Cl₆, with a catalyst selected from the group of ammonium halides, tertiary organic amines, quaternary ammonium and phosphonium halides at a temperature of 120° C. to 250° C. into higher silanes than Si₂Cl₆ is described. Tetrabutyl phosphonium chloride for example, is used as a catalyst. That document, however, does not provide any more precise details on the structures or product compositions of the perchlorinated polysilanes obtained.

A disadvantage of the known process for the preparation of perhalogenated oligosilyl and polysilyl anions or oligosilanes and polysilanes is that it does not enable the targeted and systematic preparation of linear, cyclic and cage-type silane compounds. Either mixtures of a wide variety of oligosilanes and polysilanes are obtained by these processes, the structures and compositions of which cannot be determined precisely, or the processes only permit the production of a single oligosilane or polysilane, usually with a low yield. Furthermore, the production of compounds of this kind frequently requires high temperatures and a high energy input.

### SUMMARY

It is the object of the present invention to provide a process for systematically preparing linear, cyclic and/or cage-type perhalogenated oligosilyl and polysilyl anions which overcomes the disadvantages known from the state of the art. In particular, it is intended to be possible with the process to achieve a systematic structure of linear, cyclic and cage-type perhalogenated oligosilyl and polysilyl anions which is controllable in preparative respects, and hence also to produce individual compounds in a targeted manner at comparatively low temperatures and with high yields. In addition, proceeding from the oligosilyl and polysilyl anions, it is intended to ensure access, in a manner that is simple in preparative respects, to the corresponding uncharged oligosilane and polysilane compounds. A further object is to provide corresponding oligosilyl and polysilyl anions.

The first object is achieved by a process for the preparation of linear, cyclic and/or cage-type perhalogenated oligosilyl and polysilyl anions by reacting perhalogenated monosilanes, oligosilanes or polysilanes with organosubstituted ammonium and/or phosphonium halides at temperatures ranging from −80° C. to 85° C., preferably −80° C. to 60° C.

According to the invention, the process can produce linear, cyclic or cage-type perhalogenated oligosilyl or polysilyl anions separately from one another, depending on the production conditions. Depending on the production conditions, it is, however, also conceivable that linear, cyclic and cage-type anions can be produced simultaneously side by side, which can then be separated from one another.

In the present invention, perchlorinated or perbrominated oligosilyl and polysilyl anions are preferably used.

Among the ammonium and/or phosphonium halides used, the halogen is likewise preferably F, Cl and/or Br. The ammonium and/or phosphonium halides are preferably completely organosubstituted, i.e. for example [R₄N]X with X=halide, preferably Cl and/or Br. As the organosubstituent, it is preferable to select aryl, alkyl, alkenyl and the like and mixed organo-substituents. It is particularly preferable to use ethyl, butyl and phenyl, also in a mixed substitution.

In the context of the present invention, “oligosilyl anions” and “oligosilanes” are understood to mean compounds with two to five silicon atoms.

In addition, “polysilyl anions” and “polysilanes” in the context of the present invention are understood to mean compounds with more than five silicon atoms.

For the purposes of the invention, “perhalogenated oligosilyl and polysilyl anions” are mainly or completely substituted with halogen atoms. In the context of the present invention, trichlorosilane in particular should preferably be understood as a perchlorinated monosilane.

In the context of the present invention, “linear oligosilyl and polysilyl anions” are unbranched compounds, whereas “cyclic oligosilyl and polysilyl anions” are unsubstituted and Si-substituted monocyclic oligosilyl and polysilyl anions. “Cage-type polysilyl anions” for the purposes of the invention are unsubstituted and Si-substituted polycyclic polysilyl anions.

According to the invention, there is a stoichiometric ratio of perhalogenated monosilanes, oligosilanes or polysilanes and organosubstituted ammonium and/or phosphonium halides preferably in a range from 50:1 to 1:5.

One embodiment is characterised by the fact that an additional Lewis base, preferably amine and/or phosphane is added.

In addition, it is preferably contemplated that the process is carried out in a temperature range from −80° C. to −30° C. At such low temperatures, it is mainly linear perhalogenated oligosilyl and polysilyl anions which are obtained.

In a further preferred embodiment, the process is carried out in a temperature range from −10° C. to 60° C., preferably 0° C. to 30° C., even more preferably at room temperature, in order to obtain cyclic perhalogenated polysilyl anions.

Also, it is preferable that the process is carried out in a temperature range from 0° C. to 85° C., preferably 0° C. to 60° C., preferably 10° C. to 30° C., even more preferably at room temperature, in order to obtain cage-type perhalogenated polysilyl anions if, in addition, monochlorosilanes or oligochlorosilanes or perhaps polychlorosilanes are reacted.

In addition, it is preferable in accordance with the invention that the stoichiometric ratio of perhalogenated monosilanes, oligosilanes or polysilanes and organosubstituted ammonium and/or phosphonium halides should be in a range from 50:1 to 1:1, preferably 40:1 to 1:1, even more preferably 10:1 to 1:1.

In a further preferred embodiment, a sub-stoichiometric amount of organosubstituted ammonium and/or phosphonium halides is added. Optionally, a catalytic amount of an amine can be added.

It is also preferable that the organosubstituted ammonium and/or phosphonium halides are the corresponding chlorides and bromides, even more preferably [nBu₄N]Cl, [Et₄N]Cl, [Ph₄P]Cl and [nBu₄P]Cl. Ethyl is likewise particularly preferable as an organosubstituent.

It is preferable that the monosilane is trichlorosilane, that the perhalogenated oligosilanes are preferably Si₂X₆, Si₃X₈, Si₄X₁₀ and Si₅X₁₂, X being selected from chlorine and bromine, and that the perhalogenated polysilanes are preferably polysilanes of the formula X₃Si—(SiX₂)ₙ—SiX₃, X being selected from chlorine, bromine, fluorine and iodine and n being 3 to 20. It is likewise conceivable that in the perhalogenated oligosilanes and polysilanes, X can be partially replaced by H.

Perhalogenated polysilanes for the preparation of the oligosilyl and polysilyl anions can preferably be created by plasma chemistry. Processes for the preparation of perhalogenated polysilanes are known in the art.

The process of the invention is preferably carried out using an organic solvent.

According to the invention, the solvent is preferably benzene, chlorobenzene, 1,2-dichloroethane and/or dichloromethane, though preferably dichloromethane.

The second object is achieved in accordance with the invention by oligosilyl and polysilyl anions or their H-substituted uncharged derivatives, which are obtainable by a process according to the invention.

It is preferable that the linear perhalogenated oligosilyl and polysilyl anions are [Si₃X₉]⁻, [Si₃X₁₀]²⁻, [Si₄X₁₁]⁻ and [Si₆X₁₅]⁻, X being selected from chlorine, bromine, iodine and/or fluorine, preferably chlorine or bromine.

Futhermore, it is preferable that the cyclic perhalogenated oligosilyl and polysilyl anions are [Si₆X₁₄]²⁻, [(SiX₃)Si₆X₁₃]²⁻, [1,1-(SiX₃)₂Si₆X₁₂]²⁻, [1,4-(SiX₃)₂Si₆X₁₂]²⁻, i.e. isomers of the sum formula [Si₈X₁₈]²⁻, X being selected from chlorine, bromine, iodine and/or fluorine, preferably chlorine or bromine.

Apart from that, it may preferably be contemplated that the cage-type perhalogenated poly-silyl anion is [Si₃₂X₄₅]⁻, X being selected from chlorine, bromine, iodine and/or fluorine, preferably chlorine or bromine.

It is also in accordance with the invention that the perhalogenated oligosilyl and polysilyl anions are converted into the corresponding uncharged H-substituted linear, cyclic and cage-type perhalogenated oligosilanes and polysilanes by means of additional hydrogenation.

It has surprisingly been found that in the process of the invention, the use of simple halide ions, preferably chloride ions, prepared by means of the organosubstituted ammonium and/or phosphonium halides, as donors, depending on the temperature, make the systematic production of perhalogenated oligosilyl and polysilyl anions possible. The complete range of linear, cyclic and cage-type perhalogenated oligosilyl and polysilyl anions can be obtained in a targeted manner in this way at comparatively low temperatures and with high yields. The perhalogenated oligosilyl and polysilyl anions obtained by the process of the invention can also be isolated and characterised beyond doubt by means of monocrystal X-ray structural analysis. Furthermore, the perhalogenated oligosilyl and polysilyl anions obtained can be converted into the corresponding uncharged H-substituted oligosilanes and polysilanes in a manner that is simple in preparative respects, e.g. by means of hydrogenation with conventional hydrogenation reagents such as LiAlH₄ or DIBAL-H. This results in improved solubility of the compounds in all the standard solvents, which subsequently permits refunctionalisation to the uncharged molecular and also halogenated compounds.

Building on the polarity of the Si-halogen bond, in this case especially the Si—Cl and Si—Br bond polarity, an interesting and far-reaching downstream chemistry can be carried out from the molecular products, such as substitution reactions, incorporation into polymer backbones, separation reactions, doping, ring-opening reactions followed by the formation of higher oligosilanes and polysilanes etc., leading via defined substitution products to materials with polysilane basic structures, amorphous silicon and new inorganic conductive polymers with Si-Si binding units. In this way, the targeted preparation of organo, amine and oxygen-substituted oligosilanes and polysilanes becomes possible, which are not—or only poorly—accessible via alternative synthesis routes.

### BRIEF DESCRIPTION OF THE DRAWINGS

Further features and advantages of the invention will become clear from the following description, schematic drawings and example embodiments. There,

FIG. 1 shows a chloride-induced mechanism for forming [Si₃C1₉]⁻ from Si₂C1₆ and the formation of the oligosilyl anion [Si₄Cl₁₁]⁻.

FIG. 2 shows a putative chain-propagation mechanism (left) and intramolecular cyclisation of a cyclic polysilyl anion [Si₆Cl₁₄]²⁻ (right).

FIG. 3 shows reaction routes, which result in the formation of experimentally observed species N⁻ and Q⁻.

FIG. 4 shows reaction routes, which result in the formation of 6-membered ring species.

FIG. 5 shows a molecular crystal structure of [nBu₄N][Si₃Cl₉] in the solid state, the cation not being shown.

FIG. 6 shows a molecular crystal structure of [nBu₄N]₂[Si₃Cl₁₀] in the solid state, the cation not being shown.

FIG. 7 shows a molecular crystal structure of [nBu₄N][Si₄C1₁₁] in the solid state, the cation not being shown.

FIG. 8 shows a molecular crystal structure of [nBu₄N][Si₆Cl₁₅] in the solid state, the cation not being shown.

FIG. 9 shows a molecular crystal structure of [nBu₄N]₂[Si₆Cl₁₄] in the solid state, the cation not being shown.

FIG. 10 shows a molecular crystal structure of [Ph₄P]₂[(Cl₃Si)Si₆Cl₁₃] in the solid state, the cation not being shown.

FIG. 11 shows a molecular crystal structure of [nBu₄P]₂[1,1-(Cl₃Si)₂Si₆Cl₁₂] in the solid state, the cation not being shown.

FIG. 12 shows a molecular crystal structure of [Ph₄P]₂[1,4-(Cl₃Si)₂Si₆Cl₁₂] in the solid state, the cation not being shown.

FIG. 13 shows a molecular crystal structure of [nBu₄N][Si₃₂Cl₄₅] in the solid state, the cation not being shown.

FIG. 14 shows a molecular crystal structure of [Et₄N][Si₃₂Cl₄₅] in the solid state, the cation not being shown.

FIG. 15 shows a simulated X-ray powder diffractogram (bottom), calculated from the monocrystal data of [nBu₄N]₂[Si₃Cl₁₀], and an associated X-ray powder diffractogram measured experimentally (top).

FIG. 16 shows a simulated X-ray powder diffractogram (bottom), calculated from the monocrystal data of [nBu₄N]₂[Si₆Cl₁₄], and an associated X-ray powder diffractogram measured experimentally (top).

### DETAILED DESCRIPTION

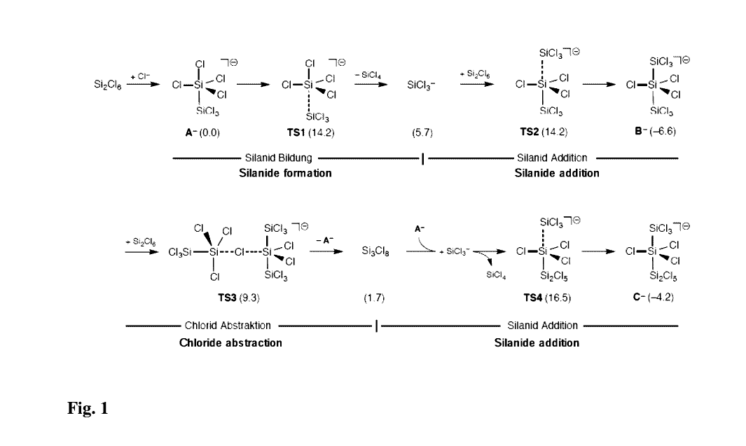

FIG. 1 shows the chloride-induced mechanism for forming [Si₃Cl₉]⁻ from Si₂Cl₆ and the formation of the higher substituted oligosilyl anion [Si₄Cl₁₁]⁻ by the subsequent formation reaction. Relative energies in FIG. 1 are stated in kcal mol⁻¹. On the basis of experimental results, and supported mechanistically by DFT calculations, linear oligosilyl anions are built up systematically, it being possible to control the reaction routes experimentally. The results of the quantum mechanical and experimental mechanistic investigations on the basis of density functional theory, X-ray structural analysis and ²⁹Si-NMR measurements as a function of varying temperatures suggest the following reaction route: the addition of chloride anions to Si₂Cl₆, first gives rise to the pentavalent adduct A⁻, which, with a low barrier, decomposes, forming SiCl₄ and [SiCl₃]⁻. The silyl anion [SiCl₃]⁻, likewise with a low barrier, subsequently adds to a further Si₂Cl₆ molecule, forming the higher anion B⁻, which was characterised by X-ray crystallography at low temperatures. After chloride abstraction by Si₂Cl₆, this leads to the intermediary formation of the higher trisilane Si₃Cl₈. The A⁻ formed in the process likewise decomposes, as described above, into SiCl₃⁻, which for its part adds to the Si₃Cl₈, forming the higher anion C⁻, which is the perhalogenated oligosilyl anion [Si₄Cl₁₁]⁻.

These corresponding elementary steps can be combined in the sense of a preparation cycle, which leads to the formation of higher oligosilyl and polysilyl anions.

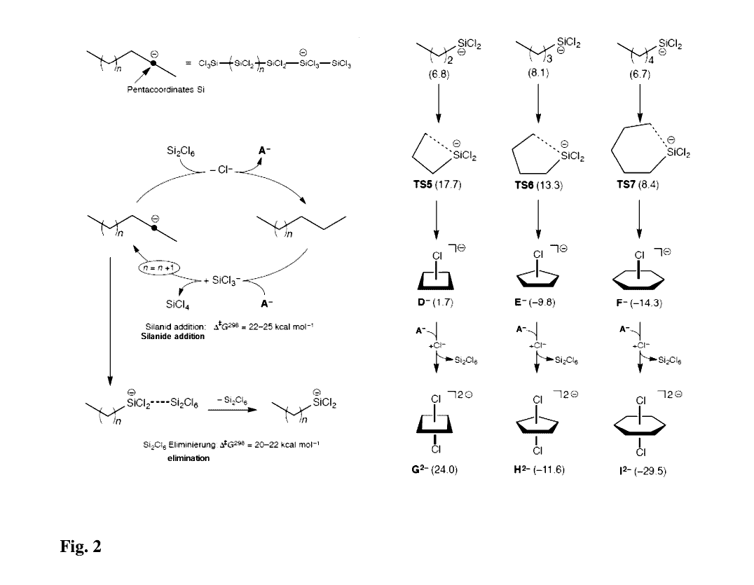

FIG. 2 (left) shows a corresponding putative chain-propagation mechanism with chain-propagation and termination steps. The chain termination is kinetically competitive with the chain-propagation and leads to the formation of terminal higher silanide anions with Si₂Cl₆ being cleaved off. The formation of the unsubstituted cyclic [Si₆Cl₁₄]²⁻ can be explained in this way by, for example, an intramolecular cyclisation reaction of higher terminal silanide anions with a chain length of n=4 and subsequent chloride addition. This reaction is heavily favoured thermodynamically compared to cyclisation reactions of shorter silanide anions with n=2 and n=3.

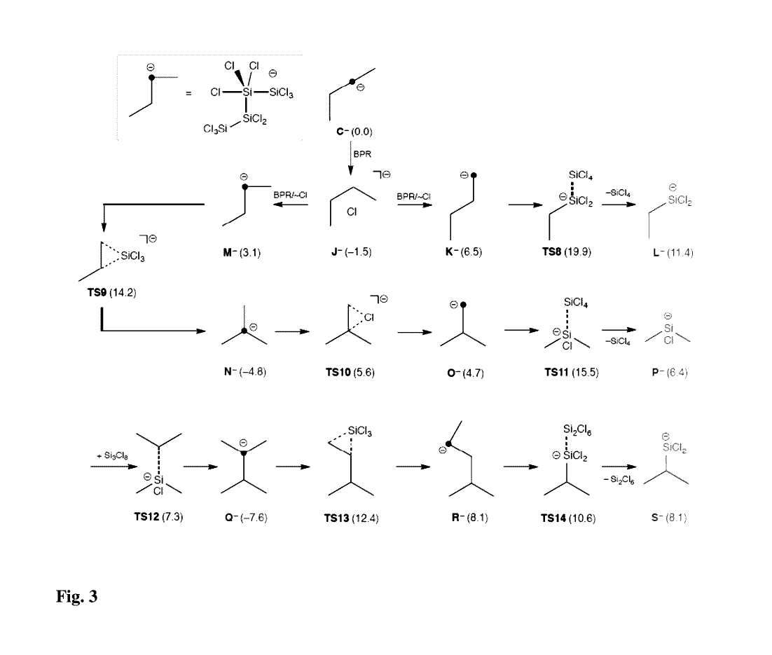

The formation of the experimentally observed dianionic perchlorosilacyclohexane chloride adducts and the SiCl₃ substitution patterns found can be explained by an alternative formation mechanism. As shown in FIG. 3, the anion C⁻ can undergo intramolecular isomerisation steps (Berry pseudorotation, BPR, chloride and silyl displacements) with moderate barrier heights. In the process, the silanide anions P⁻ and S⁻ are formed, or in a competing reaction L⁻ is formed. Along the reaction routes shown, the branched oligosilyl anions N⁻ and Q⁻ appear which were demonstrated experimentally at low temperatures, which supports the proposed mechanism.

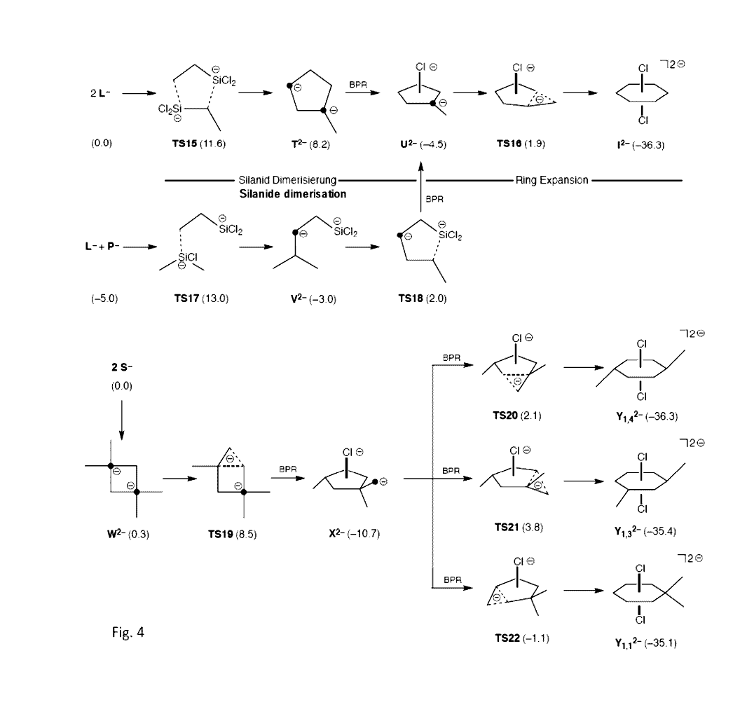

The silanide anions formed in this way can dimerise intermolecularly with very low kinetic inhibition. As shown in FIG. 4 by way of example, the formation (a) of the unsubstituted cyclic [Si₆Cl₁₄]²⁻ can be explained by the dimerisation of L⁻ or heterodimerisation of L⁻ and P⁻ and (b) the doubly silyl-substituted cyclic dianions can be explained by dimerisation of S⁻. All the routes shown involve the intermediary formation of five-membered ring systems which isomerise to the thermodynamically highly preferred six-membered by means of intra-molecular ring enlargement steps with a low barrier to ring systems. The formation of the experimentally observed silyl substitution patterns in the compounds [1,y-(SiCl₃)₂(Si₆Cl₁₂)]²⁻ (y=1, 3, and 4) can easily be explained in this figure by ring enlargement steps in different positions on the five-membered cyclic species X² formed after the dimerisation of S⁻ (FIG. 4 bottom right).

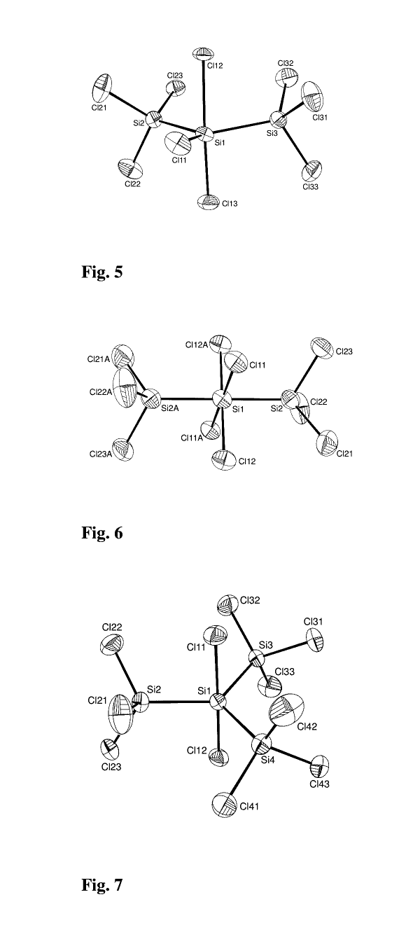

FIGS. 5-12 show the molecular crystal structures of linear and cyclic perchlorinated oligosilyl and polysilyl anions in the solid state, the corresponding cations not being shown.

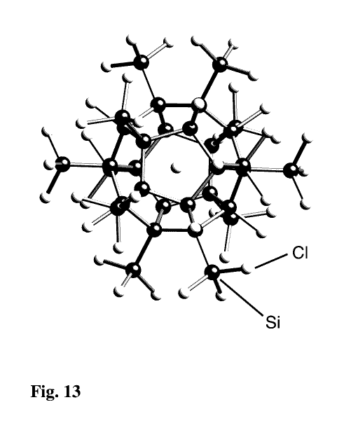

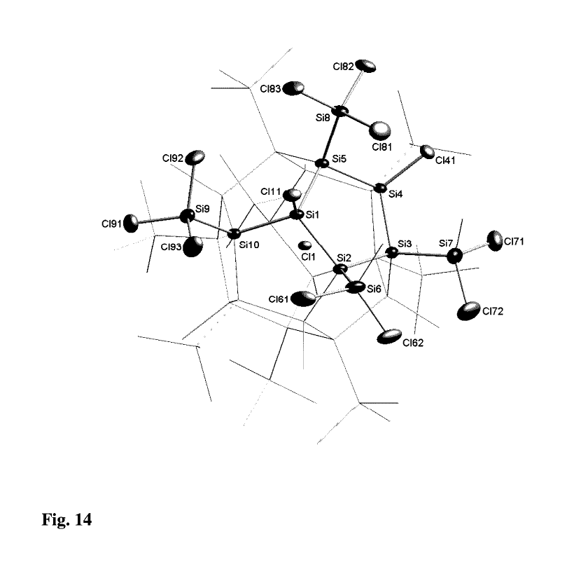

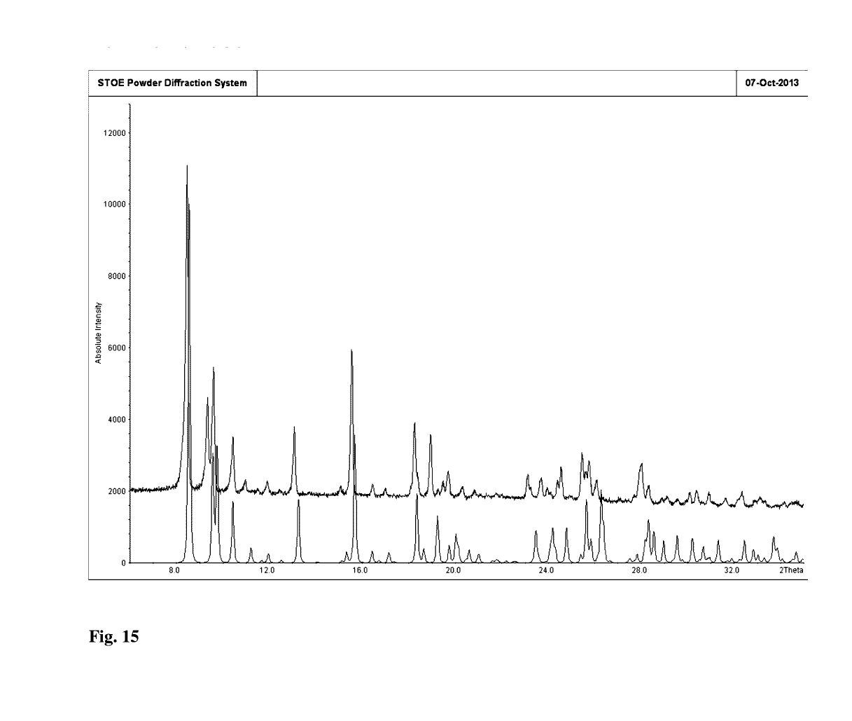

FIGS. 13 and 14 show the molecular crystal structures of [nBu₄N][Si₃₂Cl₄₅] and [Et₄N][Si₃₂Cl₄₅] in the solid state, the corresponding cations not being shown. The crystals were measured with a STOE IPDS-II diffractometer. [Et₄N][Si₃₂Cl₄₅] crystallises from CH₂Cl₂ together with 2 equivalents of SiCl₄ in the monocline space group C2/m ([Et₄N][Si₃₂Cl₄₅].2 SiCl₄). All the methylene groups of the cation are disordered over two equally occupied positions. Two Cl atoms of the SiCl₄ molecule are likewise disordered. For reasons of greater clarity, FIG. 14 only shows the asymmetrical unit as an ORTEP plot. The oscillation ellipsoid are shown with a probability of 30%. [nBu₄N][Si₃₂Cl₄₅] crystallises from CH₂Cl₂ in the hexagonal space group P6₃/m (a=b=15.016(2) Å, c=28.130(6) Å, α=β=90°, γ=120°, V=5493(1) Å³). This cage-type polysilyl anion is a Si₂₀ dodecahedron substituted with twelve SiC1₃ groups, with endohedrally coordinated chloride anions. This perchlorinated polysilyl anion can be regarded as the provisional end product of a systematic formation reaction.

FIG. 15 shows a simulated X-ray powder diffractogram (bottom), calculated from the mono-crystal data of [nBu₄N]₂[Si₆Cl₁₄], and an X-ray powder diffractogram measured experimentally and taken from the raw product obtained (top). A comparison between the two powder diffractograms shows the phase purity of the raw [nBu₄N]₂[Si₃Cl₁₀] product obtained. The monocrystal data were obtained at a temperature of −100° C., while the powder data were collected at room temperature.

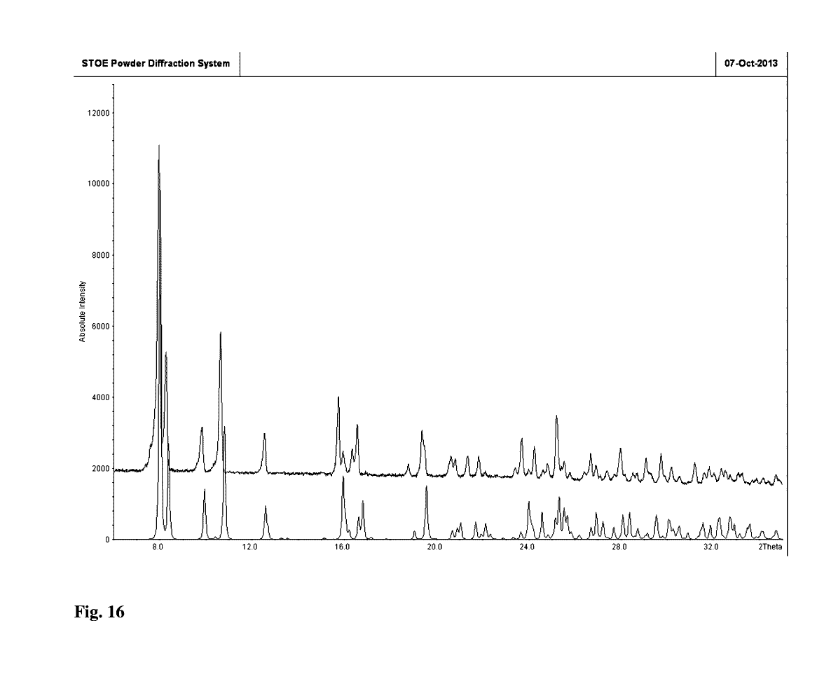

FIG. 16 shows a simulated X-ray powder diffractogram (bottom), calculated from the mono-crystal data of [nBu₄N]₂[Si₆Cl₁₄], and an X-ray powder diffractogram measured experimentally and taken from the raw product obtained (top). A comparison between the two powder diffractograms shows the phase purity of the raw [nBu₄N]₂[Si₆Cl₁₄] product obtained. The monocrystal data were obtained at a temperature of −100° C., while the powder data were collected at room temperature.

### EXAMPLE EMBODIMENTS

### 1. General Working Conditions

All the reactions were carried out under dry argon or nitrogen. CH₂Cl₂ was dried over CaH₂ and freshly distilled before use; CD₂Cl₂ was dried over a molecular sieve in the presence of silver foil (4 Å). [D₈]THF was dried over sodium. Si₂Cl₆, [nBu₄N]Cl, [Et₄N]Cl, [Ph₄P]Cl and [nBu₄P]Cl are commercially available; the chlorides were dried in a vacuum at room temperature for 2 d. [nBu₄N]Cl usually contains traces of KCl; it was therefore dissolved in CH₂Cl₂ as a matter of principle, and the insoluble KCl was removed by filtration. The temperature for low-temperature experiments was controlled with a Haake EK 90 cryostat. ²⁹Si NMR spectra were recorded with a Bruker Avance III HD 500 MHz spectrometer; the spectra were calibrated against the external standard SiMe₄ (δ(²⁹Si)=0). X-ray powder data were collected with a STOE STADI P diffractometer (linear PSD; CuKα₁-radiation (λ=1.5406 Å)). Elemental analyses (EA) were carried out by the microanalytical laboratory of the Goethe University Frankfurt. LDI-MS spectra were recorded with a MALDI LTQ Orbitrap XL.

### 2. Synthesis of Linear Perhalogenated Oligosilyl and Polysilyl Anions

### 2.1. Synthesis of [nBu₄N][Si₃Cl₉] and [nBu₄N][Si₆Cl₁₅]

2 Si₂Cl₆+[nBu₄N]Cl→[nBu₄N][Si₃Cl₉]+SiCl₄

Scheme 1. Reaction equation for the synthesis of [nBu₄N][Si₃Cl₉]. For the experiment, a stoichiometric ratio of Si₂Cl₆:[nBu₄N]Cl=40:1 was chosen.

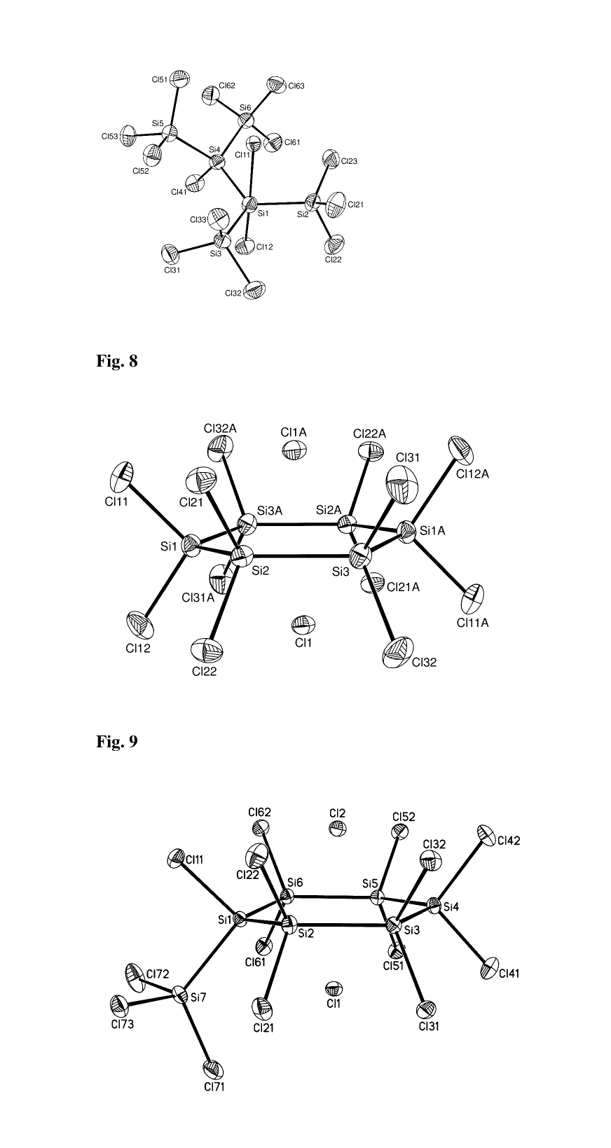

Si₂Cl₆ (5.0 mL, 7.8 g, 29 mmol) was added at −50° C. to a solution of [nBu₄N]Cl (0.20 g, 0.72 mmol) in CH₂Cl₂ (15 mL) without stirring. In the process, Si₂Cl₆ solidified directly upon addition and only dissolved slowly in the solvent. The reaction mixture was held at −50° C. for 24 h with the aid of a cryostat, during which time colourless acicular monocrystals of [nBu₄N][Si₃Cl₉] (FIG. 5) formed. No other residue was observed in the clear mother liquor. Some of the air-sensitive and moisture-sensitive crystals were removed from the cold (−50° C.) reaction flask, selected under a stream of cold nitrogen and examined radiographically. After [nBu₄N][Si₃Cl₉] had been identified, the solution was warmed up to −40° C. with the remaining crystals and stored at that temperature. After 12 d, the shape of the crystals, although still acicular, had changed visibly, and their number had increased. The crystals were isolated in the cold and their structure determined with X-ray diffractometry on the monocrystal as [nBu₄N][Si₆Cl₁₅] (FIG. 8).

### 2.2. Synthesis of [nBu₄N]₂[Si₃Cl₁₀]

2 Si₂Cl₆+2[nBu₄N]Cl→[nBu₄N]₂[Si₃Cl₁₀]+SiCl₄

Scheme 2. Reaction equation for the synthesis of [nBu₄N]₂[Si₃Cl₁₀].

Si₂Cl₆ (5.0 mL, 7.8 g, 29 mmol) was added at −78° C. to a solution of [nBu₄N]Cl (8.1 g, 29 mmol) in CH₂Cl₂ (20 mL) without stirring. Si₂Cl₆ solidified directly upon addition and only dissolved slowly. The reaction mixture was held at −78° C. for 1 week with the aid of a cryostat. During that time, a large number of crystals formed in the upper part of the solution. A monocrystal was isolated and identified radiographically as [nBu₄N]2[Si₃Cl₁₀] (FIG. 6). The phase purity of the raw product was established with the aid of X-ray powder diffractometry (FIG. 15). Yield of crystalline material: 6.5 g (49%). EA (%) calculated for C₃₂H₇₂Cl₁₀ N₂Si₃ [923.69]: C 41.61, H 7.86, N 3.03; C 41.32, H 7.96, N 3.34.

### 2.3. Synthesis of [nBu₄N][Si₄Cl_{11]}

3 Si₂Cl₆+[nBu₄N]Cl→[nBu₄N][Si₄Cl₁₁]+2 SiCl₄

Scheme 3. Reaction equation for the synthesis of [nBu₄N][Si₄Cl₁₁]. For the experiment, a stoichiometric ratio of Si₂Cl₆:[nBu₄N]Cl=10:1 was chosen.

Si₂Cl₆ (9.0 mL, 14 g, 52 mmol) was added at −50° C. to a solution of [nBu₄N]Cl (1.5 g, 5.4 mmol) in CH₂Cl₂ (30 mL) with gentle stirring. The reaction mixture was held at −50° C. for 4 d without stirring. After that, it was possible to isolate colourless, air-sensitive and moisture-sensitive monocrystals from the reaction solution and to determine the structure of [nBu₄N][Si₄Cl₁₁] (FIG. 7).

### 3. Synthesis of Cyclic Perhalogenated Polysilyl Anions

As a matter of principle, the syntheses were carried out by adding Si₂Cl₆ to a solution of the corresponding chloride salt ([nBu₄N]Cl, [nBu₄P]Cl or [Ph₄P]Cl) in CH₂Cl₂. It was also possible to obtain chloride adducts of cyclic perchlorinated hexasilyl anions with C₆H₅Cl and C₆H₆ as solvents, but CH₂Cl₂, proved the preferable reaction medium because of its properties.

According to in situ ²⁹Si NMR spectroscopy, the formation of significant quantities of (substituted) cyclic hexasilyl anions begins between −10° C. and 0° C. Irrespective of the chloride salt used, the formation of only one (substituted) cyclic hexasilyl anion was never observed. The ratio of Si₂Cl₆ to Cl⁻ ions was varied in the range from 1:5 to 10:1. It became clear that the stoichiometry has hardly any influence on the product distribution. The formation of the relative amounts of different (substituted) cyclic hexasilyl anions in the reaction solution can, however, be influenced by the reaction temperature. Preparatively useful amounts of a specific (substituted) cyclic hexasilyl anion were obtained by fractionating crystallisation: (i) substituted cyclic hexasilyl anions crystallise before the unsubstituted cyclic hexasilyl anions as a rule. (ii) Different densities of the various crystals obtained can be used to separate the substituted rings from the unsubstituted ring: In CH₂Cl_{2,} [nBu₄N][Si₆Cl₁₄] floats on the surface, while the doubly substituted rings sink to the bottom. Larger amounts of [nBu₄N][Si₆Cl₁₄] can also be obtained by targeted tempering of a mixture of substituted cyclic hexasilyl anions. In the process, the Cl₃Si substituents are replaced by Cl substituents, and [nBu₄N][Si₆Cl₁₄] is formed in this way.

The yields relate to the amounts of Si₂Cl₆ used, on the assumption that each Si₂Cl₆ molecule contributes precisely one SiCl₂ component to the formation of the product molecules.

### 3.1. Synthesis of [nBu₄N]₂[1,1-(SiCl₃)₂Si₆Cl₁₂] as the main component in a mixture with other isomers of [nBu₄N]₂[Si₈Cl₁₈]

Si₂Cl₆ (9.30 mL, 14.5 g, 53.9 mmol) was added at room temperature to a solution of [nBu₄N]Cl (5.0 g, 18 mmol) in CH₂Cl₂ (20 mL) without stirring. The clear reaction mixture turned slightly yellowish. After 12 h at room temperature, the solution was colourless again and after a few days crystals began to precipitate. The crystalline material was removed from the solution after a total of 20 d. Total yield of [nBu₄N]₂[Si₈Cl₁₈]: 2.96 g (33%). EA (%) calculated for C₃₂H₇₂Cl₁₈N₂Si₈ [1347.74]: C 28.52, H 5.38, N 2.08; C 28.66, H 5.68, N 1.71. ²⁹Si NMR data for a solution of a number of selected monocrystals of [nBu₄N]₂[1,1-(SiCl₃)₂Si₆Cl₁₂] (99.4 MHz, CD₂Cl₂, 298 K): δ=11.4 (¹J(Si,Si)=73 Hz, ²J(Si,Si)=14 Hz, 2Si; SiCl₃), −22.3 (¹J(Si,Si)=148 Hz, ²J(Si,Si)=29 Hz, ²J(Si,Si)=14 Hz, 2Si; Si-2), −24.8 (²J(Si,Si)=29 Hz, 1Si; Si-4), −27.3 (²J(Si,Si)=21 Hz, 2Si; Si-3), −52.5 ppm (¹J(Si,Si)=148 Hz, ¹J(Si,Si)=73 Hz, ²J(Si,Si)=21 Hz, 1Si; Si-1). Note: The ²⁹Si resonances were assigned on the basis of (i) relative integral surfaces and (ii) Si,Si coupling constants measured between the resonances of the ²⁹Si satellites. The ²⁹Si satellites needed for determining ¹J(Si,Si) coupling constants between Si-2/Si-3 and Si-3/Si-4 are not sufficiently well resolved to obtain reliable values.

X-ray structural analysis of numerous monocrystals, selected from the crystalline material obtained in all cases demonstrated the constitution of the anion [1,1-(SiCl₃)₂Si₆Cl₁₂]²⁻.

### 3.2. Synthesis of [nBu₄N]₂[Si₆Cl₁₄]

Method A: As a second product from the synthesis of [nBu₄N]₂[1,1-(SiCl₃)₂Si₆Cl₁₂] (see above), it was possible to isolate crystals of [nBu₄N]₂[Si₆Cl₁₄] from the mother liquor after the crystals of the substituted Si₆ rings had been separated. After a storage period of 60 d, [nBu₄N]₂[Si₆Cl₁₄] was obtained with a yield of 3.50 g (34%). It was possible to measure the crystals with X-ray diffractometry (FIG. 9); the phase purity of the raw product was confirmed with X-ray powder diffractometry (FIG. 16) and ²⁹Si NMR spectroscopy^{[1, 2]}. ²⁹Si NMR of [nBu₄N]₂[Si₆Cl₁₄] (99.4 MHz, CD₂Cl₂, 298 K): δ=−21.7 ppm (SiCl₂).

Method B: HSiCl₃ (6.7 mL, 9.0 g, 66 mmol) was placed at room temperature, with stirring, in a screw-top jar with a solution of [nBu₄N]Cl (6.1 g, 22 mmol) and Bu₃N (8.6 mL, 12 g, 65 mmol) in CH₂Cl₂ (20 mL). The clear solution was stirred for 10 d. After that C₆H₆ (15 mL) was added, whereupon crystals settled at the bottom of the solution. The crystals were isolated after 20 d and identified as [nBu₄N]₂[Si₆Cl₁₄] with X-ray structural analysis. 3.5 g product were isolated (yield 27%).

### 3.3. Synthesis of [Ph₄P]₂[(Cl₃Si)Si₆Cl₁₃] and [Ph₄P1]₂[Si₆Cl_{14]}

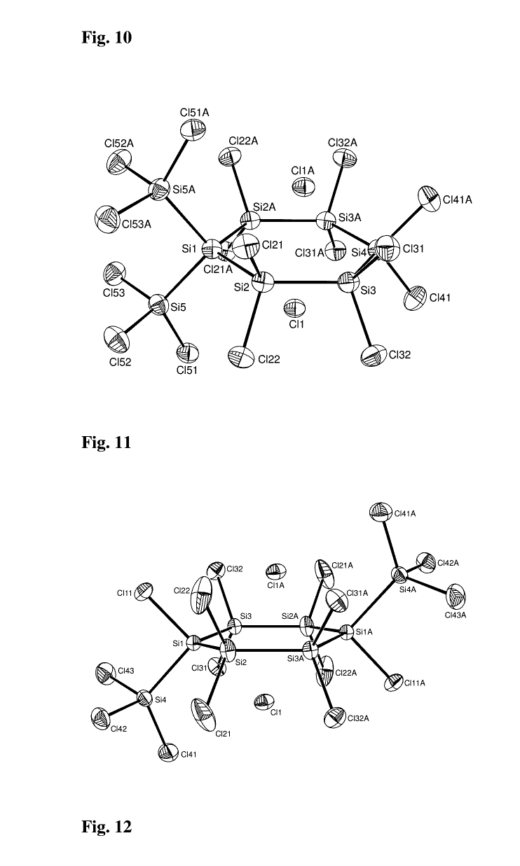

The reaction was carried out in a flask divided into two equal parts by a glass wall. A solution of Si₂Cl₆ (1.0 mL, 1.6 g, 6.0 mmol) in CH₂Cl₂ (1 mL) was filled into one half, the other side was filled with a solution of [Ph₄P]C1 (0.60 g, 1.6 mmol) in CH₂Cl₂ (2 mL). The sealed flask was stored under nitrogen at room temperature. After 7 d, small crystals were obtained on the side of the salt. X-ray diffractometry showed the structure of [Ph₄P]₂[Si₆Cl₁₄]. After all the crystals had been removed, the flask was sealed again in order to continue the gas diffusion process. During the following 50 d, large crystals of [Ph₄P]₂[(Cl₃Si)Si₆Cl₁₃] formed (FIG. 10) in the half of the flask filled with [Ph₄P]Cl solution; the other side of the flask was almost empty at this time. ²⁹Si-NMR data of a single dissolved crystal of [Ph₄P]₂[(Cl₃Si)Si₆Cl₁₃] (99.4 MHz, [D₈]THF, 298 K): δ=9.9 (1Si; SiCl₃), −20.6 (1Si; Si-4), −21.6 (2Si; Si-2 or 3), −22.5 (2Si; Si-2 or 3), −49.0 ppm 1Si; Si-1). Note: The ²⁹Si resonances were assigned on the basis of the relative integral surfaces and a comparison with chemical shifts in similar compounds; ²⁹Si satellites were not dissolved.

### 3.4. Synthesis of [nBu₄P]₂[1,1-(Cl₃Si)₂Si₆Cl₁₂] and [nBu₄P]₂[Si₆Cl₁₄]

Si₂Cl₆ (0.20 mL, 0.31 g, 1.2 mmol) was added to a frozen solution of [nBu₄P]Cl (86 mg, 0.29 mmol) in CD₂Cl₂ (0.5 mL) in an NMR tube at −196° C. The NMR tube was sealed in a vacuum, and the reaction solution was rapidly raised to −60° C.; after that, the solution was slowly warmed up to room temperature, in the process of which a white solid precipitated from a clear solution. Crystals selected from that solid showed the structures of [nBu₄P]₂[1,1-(Cl₃Si)₂Si₆Cl₁₂] (FIG. 11) and [nBu₄P]₂[Si₆Cl₁₄].

### 3.5. Synthesis of [Ph₄P]₂[1,4-(Cl₃Si)₂Si₆Cl₁₂] and [Ph₄P]₂[Si₆Cl₁₄]

Si₂Cl₆ (0.17 mL, 0.27 g, 1.0 mmol) was added to a solution of [PhP]C1 (0.15 g, 0.40 mmol) in CD₂Cl₂ (0.5 mL) in an NMR tube. The NMR tube was sealed in a vacuum, and stored at room temperature. Crystals were obtained after a few days and showed the crystal structures of [Ph₄P]₂[1,4-(Cl₃Si)₂Si₆Cl₁₂] (FIG. 12) and [Ph₄P]₂[Si₆Cl₄].

4. Synthesis of Cage-Type Perhalogenated Polysilyl Anions

### 4.1. Synthesis of [nBu₄N][Si₃₂Cl₄₅]

Method A: Si₂Cl₆ (5 mL, 7.8 g, 29 mmol) was placed as a layer in a flask at 0° C., over a colourless solution of [nBu₄N]Cl (0.8 g, 2.9 mmol) and Bu₃N (0.5 mL, 0.37 g, 3.6 mmol) in CH₂Cl₂ (5 mL). The two-phase system was raised to room temperature overnight, in which time both phases had turned a deep orange and crystals had formed at the phase boundary. After a further 24 h, the lower phase had turned brown; the two phases were separated. After three weeks, crystals were isolated from the lower phase and identified with laser-induced mass spectrometry and X-ray structural analysis as [nBu₄N][Si₃₂Cl₄₅] (FIG. 13). 100 mg product were isolated (yield 5%).

Method B: A Schott flask with a screw-top lid was filled with [nBu₄N]Cl (1.0 g, 3.6 mmol), Bu₃N (0.36 g, 1.9 mmol) and CH₂Cl₂ (9 mL) in an argon-filled glovebox. Si₂Cl₆ (6.4 mL, 10 g, 37 mmol) was added with a syringe, in one portion, at room temperature and with stirring. The initially colourless solution turned yellow. After two days of stirring, the colour had changed to brown/orange and a colourless solid had formed. The solid, substantially [nBu₄N]₂[1,1-(SiCl₃)₂Si₆Cl₁₀.2 Cl], was removed by filtration. The slightly cloudy filtrate was mixed with CH₂Cl₂ (10 mL) and the resulting clear solution was stored in a Schott flask. After about one week, colourless crystals with a hexagonal cross-section were obtained. The mother liquor was decanted and the crystals obtained were washed with CH₂Cl₂ (4×5 mL). Yield of [nBu₄N][Si₃₂Cl₄₅]: 0.70 g (29%). ²⁹Si-NMR (99.37 MHz, THF[D₈]): δ 31.1 (Si(I)), 10.3 (Si(III)), −60.4 ppm (Si(0)); LDI-MS (m/z): [M]⁻ calculated for [Si₃₂Cl₄₅]⁻, 2492.8; found 2492.8; LDI-MS (m/z): [M]⁺ calculated for [nBu₄N]⁺, 242.3; found 242.3.

Method C: The anion of the desired target substance can also be obtained proceeding from HSiCl₃ (1 ml) with Bu₃N (1 ml) and the addition of [nBu₄N]Cl without a solvent. The yield of [nBu₄N][Si₃₂Cl₄₅] is then slightly lower (approx. 10%).

### 4.2. Preparation of monocrystalline material of [nBu₄N][Si₃₂Cl₄₅] and [Et₄N][Si₃₂Cl₄₅]

X-ray crystallography on [nBu₄N][Si₃₂Cl₄₅] shows a hexagonal packing of [Si₃₂Cl₄₅]⁻ anions; [nBu₄]⁺ counter-ions and probably CH₂Cl₂/SiC1₄ molecules occupy the free space between the spherical anions. Both the cation and the solvent molecules are disordered to such an extent that no unambiguous resolution of the measured data is possible. The molecular structure of the cluster anion was ultimately solved with the aid of the SQUEEZE function of the Platon program.

In order to reduce the disordering of the cation, the synthesis was also carried out with [Et₄N]Cl as the chloride source. In the reaction, a large amount of insoluble solid formed, which is the reason why [Et₄N]⁺ as the counter-ion is somewhat less suitable for the preparative synthesis. Even so, it was possible to obtain monocrystals of high quality. With X-ray structural analysis, they could be unambiguously characterised as [Et₄N][Si₃₂Cl₄₅].2 SiCl₄ (FIG. 14).

The features of the invention disclosed in the above description, the claims and the drawings can be essential to implementing the invention in its various embodiments both individually and in any combination.

1. A process for the preparation of linear, cyclic and/or cage-type perhalogenated oligosilyl and polysilyl anions by reacting perhalogenated monosilanes, oligosilanes or polysilanes with one or more of organosubstituted ammonium and phosphonium halides at temperatures ranging from −80° C. to 85° C.

2. The process as claimed in claim 1, in which there is a stoichiometric ratio of the perhalogenated monosilanes, oligosilanes or polysilanes and the one or more organosubstituted ammonium and phosphonium halides in a range from 50:1 to 1:5.

3. The process as claimed in claim 1, wherein an additional Lewis base is added.

4. The process as claimed in claim 1, wherein it is carried out in a temperature range from −80° C. to −30° C.

5. The process as claimed in claim 1, wherein it is carried out in a temperature range from −10° C. to 85° C.

6. The process as claimed in claim 1, wherein it is carried out in a temperature range from 0° C. to 60° C., and the perhalogenated polysilane is reacted.

7. The process as claimed in claim 1, wherein the stoichiometric ratio of the perhalogenated monosilanes, oligosilanes or polysilanes and the one or more organosubstituted ammonium and phosphonium halides is in a range from 50:1 to 1:1.

8. The process as claimed in claim 1, wherein a sub-stoichiometric amount of the one or more organosubstituted ammonium and phosphonium halides is added.

9. The process as claimed in claim 1, wherein the perhalogenated oligosilyl and polysilyl anions are converted into corresponding uncharged H-substituted linear, cyclic and cage-type perhalogenated oligosilanes and polysilanes by means of subsequent hydrogenation.

10. Perhalogenated oligosilyl and polysilyl anions or their H-substituted derivatives, prepared by the process as claimed the process as claimed in claim 9.

11. Perhalogenated oligosilyl and polysilyl anions or their H-substituted derivatives, prepared by the process as claimed in the process as claimed in claim 1.

12. The process as claimed in claim 3 wherein the additional Lewis base is an amine or a phosphoane or both an amine and a phosphane.

13. The process as claimed in claim 2, wherein an additional Lewis base is added.

14. The process as claimed in claim 13 wherein the additional Lewis base is an amine or a phosphoane or both an amine and a phosphane.

15. The process as claimed in claim 2, wherein it is carried out in a temperature range from 0° C. to 60° C., and the perhalogenated polysilane is reacted.

16. The process as claimed in claim 3, wherein it is carried out in a temperature range from 0° C. to 60° C., and the perhalogenated polysilane is reacted.

17. The process as claimed in claim 1, wherein a sub-stoichiometric amount of the one or more organosubstituted ammonium and phosphonium halides is added, with the addition of a catalytic amount of an amine.
